(19) United States
(12) Patent Application Publication
(10) Pub. No.: US 2008/0161827 A1
(43) Pub. Date: Jul. 3, 2008

(54) System and method for a selectively visible medical marker

(76) Inventor: Ricky A. Frost, Tewksbury Township, NJ (US)

Correspondence Address: Matthew J. Hodulik, 9 Cross Brook Drive, Califon, NJ, 07830, US

(21) Appl. No.: 11/648,289

(22) Filed: Dec. 29, 2006

(51) Int. Cl. A61B 5/117

(52) U.S. Cl. 606/116

(57) ABSTRACT

A new approach for a system and method for producing a substantially invisible medical tissue marker is described. In certain exemplary embodiments of the invention, the medical marker will become visible only when a light (energy) source is shined thereon. The visible action of the marker is accomplished, for example, utilizing materials that are multi-photon absorbers. In another exemplary embodiment, the medical marker will become visible for a given amount of time when the tissue marker is exposed to a specific light source.

### TECHNICAL FIELD

This invention relates generally to tissue markings and more particularly to tissue markings used as medical markings.

### BACKGROUND OF THE INVENTION

In certain medical procedures, for example, radiation therapy for breast cancer, medical markings are applied to the skin as a “bulls-eye” or marking for a radiologist to target It is very important that the marks be maintained from one treatment to the next. Many oncologists argue that it is critically important for the markings to stay with the patient forever because they need to know the exact location of the initial radiation treatments in order to limit unnecessary side effects should the patient relapse.

These medical markings are applied in a like fashion to that of conventional tattoos, wherein a needle-like instrument is used to inject colored suspended pigments or dyes into the skin of the person receiving the marking. Consequently, these markings, like tattoos are permanent in nature and may be removed only by surgical techniques, lasers, microderm abrasion techniques or other invasive procedures.

It is known that the permanent application of these medical markers may at times have adverse psychological effects on a patient receiving treatment in that the medical marker serves as a constant reminder of the illness that he or she has been battling. This is true even after treatment of an illness has been discontinued, since the permanent marker is always visible.

Based on the above, there is a need for a permanent medical marker that may be selectively made visible.

### SUMMARY OF THE INVENTION

An advance is made over the prior art in accordance with the principles of the present invention that is directed to a new approach for a system and method for producing a substantially invisible medical tissue marker. In certain exemplary embodiments of the invention, the medical marker will become visible only when a light (energy) source is shined thereon. The visible action of the marker is accomplished, for example, utilizing materials that are multi-photon absorbers. In another exemplary embodiment, the medical marker will become visible for a given amount of time when the tissue marker is exposed to a specific light source.

One exemplary method of marking an exterior skin surface for marking purposes comprises the steps of locating an area on said skin surface to be marked and marking the area with a substantially colorless marking solution suitable for embedding in the skin surface. The marking solution includes one or more active compounds that appear substantially colorless and are substantially invisible to the naked eye until exposed to an excitation source. The active compounds cause a frequency change to light emitted from said excitation source and directed thereto, thereby further causing visible light to be emitted from the active compounds while the excitation source is directed at the marked area. A corresponding system includes a substantially colorless marking solution suitable for embedding in said skin surface.

Another exemplary embodiment of the invention includes a method of utilizing a medical marking comprising the steps of generally locating a marked area, where the area is marked with a marking solution embedded in tissue. The solution includes one or more active compounds that appear substantially colorless and are substantially invisible to the naked eye until exposed to an excitation source. The active compounds cause a frequency change to light emitted from the excitation source thereby altering the frequency of the light by frequency up conversion or frequency down conversion and emitting visible radiation. The method further includes the step of exciting the active compounds to cause visible radiation to emit from the marked area, thereby allowing exposure of the marked area for purposes of medical treatment.

### BRIEF DESCRIPTION OF THE DRAWING

The teachings of the present invention can be readily understood by considering the following detailed description in conjunction with the accompanying drawings, in which:

FIG. 1 shows an energy diagram for one exemplary embodiment of invisible medical marker as it is excited to a visible state;

FIGS. 2 and 3 show an exemplary illustration of the operation of an invisible medical marker according the principles of the present invention;

FIG. 4 shows a block diagram of exemplary medical equipment having an integrated excitation source; and

FIG. 5 shows an energy diagram for another exemplary embodiment of an invisible medical marker methodology as it is excited to a visible state.

### DETAILED DESCRIPTION

Exemplary embodiments of the invention will now be described while referring to the figures, several of which may be simultaneously referred to during the course of the following description.

The present invention discloses a system and method for producing a substantially invisible medical tissue marker. In the context of the present invention, the term invisible shall mean barely visible or significantly less visible than a conventional dark ink medical marking or tattoo. The tissue marker, which may identify a specific location for the application of a medical treatment, such as radiation treatment, will remain invisible until exposed to a source of electromagnetic radiation, as will further be described herein. In certain exemplary embodiments of the invention, the medical marker will become visible only when a light (energy) source is shined thereon. In another exemplary embodiment, the medical marker will become visible for a given amount of time when the tissue marker is exposed to a specific light source such as ultraviolet light.

By providing a medical tissue marker whose visibility is selectively activatable only when desired, for instance at the time of treatment, a patient will not suffer the constant reminders associated with a permanently visible marker from his or her treatment. Nor will a permanent marking be left on a person's body after treatment has been discontinued, where the patient may then wish to have the tissue marker removed by invasive procedures. Additionally, since physicians desire that medical tissue markers be left in place even after treatments are over, the medical marker of the invention can remain permanently present without psychologically impacting the patient.

This invention will provide for safer and more efficacious medical treatments, since the invention enables providing for much greater compliance with usage of permanent markers and their associated benefits. Currently, radiation therapy patients choose the less safe option of no markings because of the potential stigma of being tattooed. The present invention will increase compliance and increase the physician's ability to treat optimally because of knowing the location of historic treatments.

The invisible medical marker of the invention is achieved by applying a specialized marking solution to a desired location of the skin tissue in a similar fashion as a tattoo. In the context of the present description, the term solution shall mean a solution, suspension or even a dry powder with no carrier. In cases other than the dry powder, the marking solution is produced by including an activatable agent within a carrier, wherein the marking solution is applied to the skin using conventional medical marking tools and technology that are similar in fashion to those instruments utilized for application of tattoos.

One exemplary embodiment of a medical marker according to the present invention utilizes a tissue marking that is visible only for the time that a light source or other energy source is shone on the marking. The visible action of the marker is accomplished, for example, utilizing materials that are multi-photon absorbers. In such a case, low energy excitation photons of infrared light energy, for example, such as light produced from an infrared laser are directed so as to be incident on the medical marker. The laser emits light at a wavelength, for example, of between 0.6 and two microns wavelength, although other wavelengths may be utilized. As would be understood by persons skilled in the art, in a single particle in the marker, multiple low energy photons are absorbed and one higher energy, visible, photon is emitted. This process of producing light is known generally as frequency upconversion.

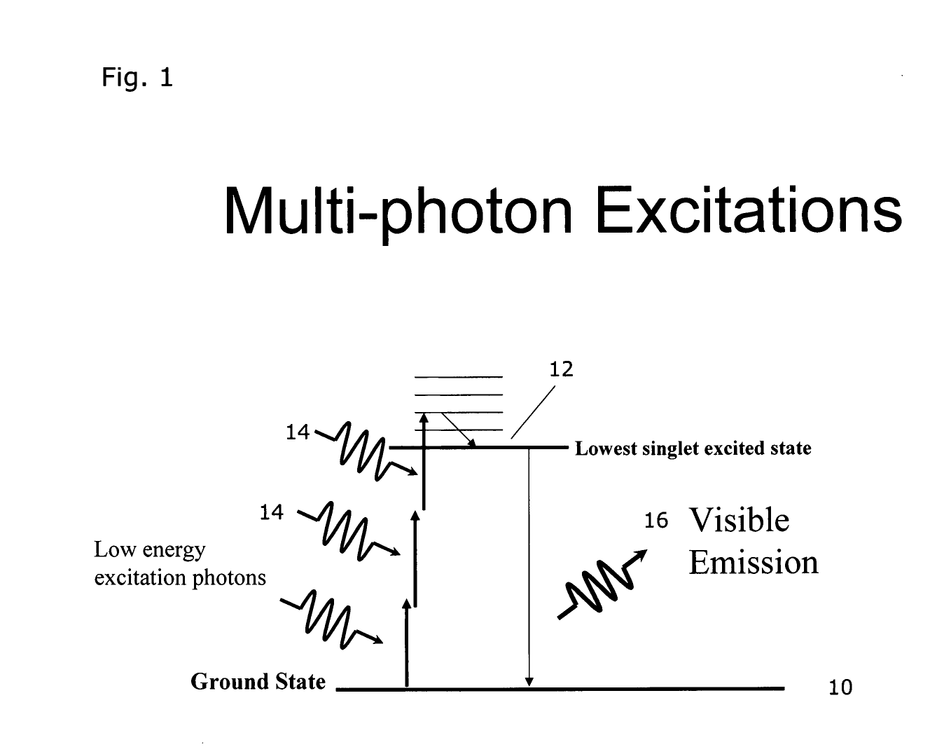

FIG. 1 shows an energy diagram that describes the manner in which multi-photon excitation materials may be utilized in order to produce a visible emission. As can be seen a material is altered from its ground state 10 to its lowest single excited state 12 by means of low energy excitation photons 14 produced from an excitation source, such as a laser. Once the excited state is reached, a visible emission 16 takes place. Examples of multi-photon excitation materials are rare earth doped frequency upconverting crystals. Such materials include Gadolinium oxides such as, Gadolinium Gallium Aluminum oxide, Gd₂Ga₂Al₂0₃, sold under the trade name of Sunstones (by Sunstone, Inc. of Allentown, N.J.). This family of rare-earth doped crystals has unique characteristics in that their spectral properties (i.e. absorption and emission) can be tailored to provide absorptions at wavelength where convenient emission sources exist (e.g., LEDs, lamps or lasers) and emissions that are easily visible to the naked eye or to cameras

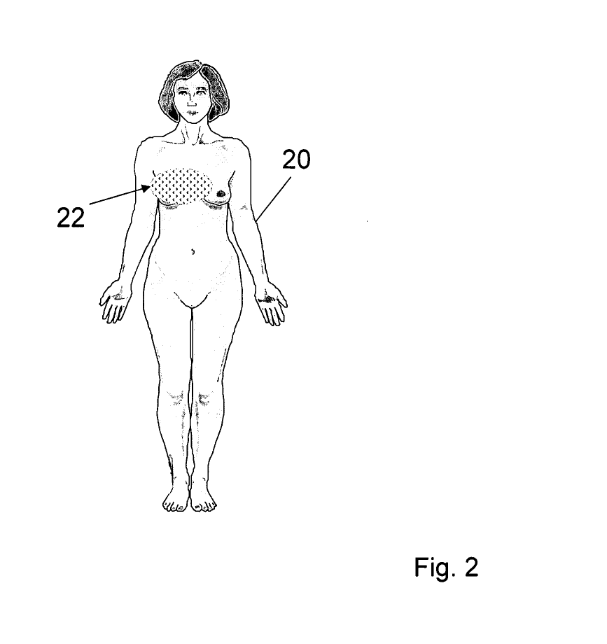

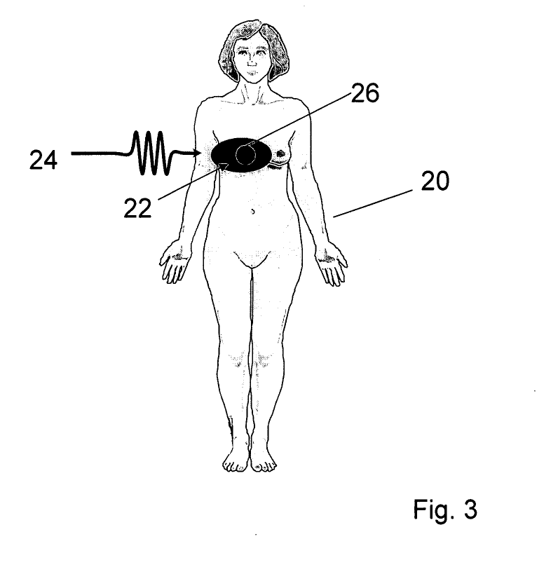

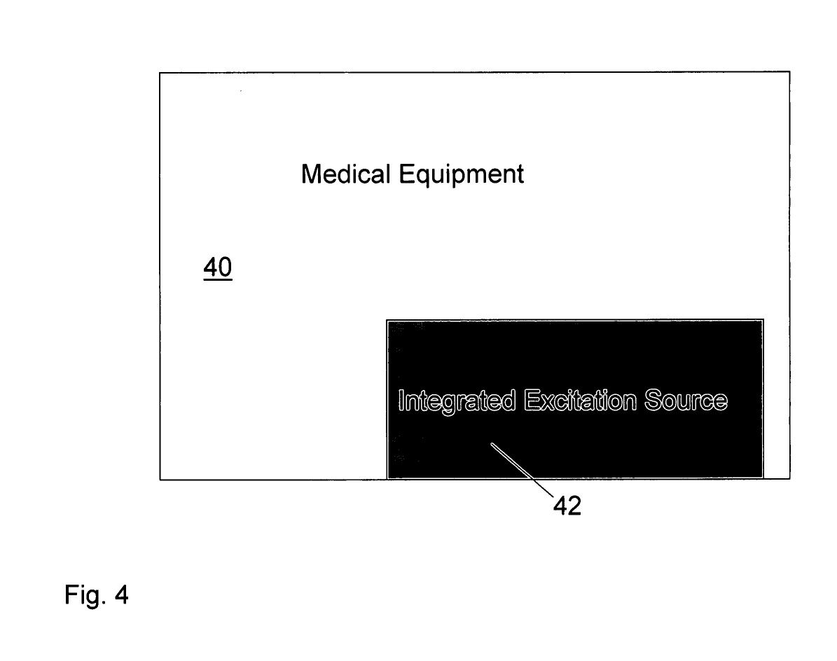

Referring to FIGS. 2 and 3, there are shown simplified diagrams illustrating the principles of one exemplary embodiment of the present invention. FIG. 2 shows a hypothetical medical patient 20 having a medical marker applied within the patterned area 22 in accordance with the techniques of the present invention to her breast area. As represented by the patterned area 22, the medical marker has been applied to the skin tissue, but remains invisible until the active agent within the inventive solution is activated. FIG. 3 shows the same patient 20 being exposed to energy 24 from an excitation source, such as a low level laser or infrared light, directed at the patterned area 22, which is the general area containing the medical marker. As can be seen, the medical marker 26 is plainly visible so as to be able to receive precisely directed treatment when the agent is activated by the energy source. At this time a medical practitioner may optionally choose to mark the activated area with a temporary marking so that treatment may continue without having to continuously activate the medically marked location. Once the energy source is removed, the marking returns to its invisible (to the naked eye) condition. As would be understood to those persons skilled in the art, the medical marking techniques described in accordance with the present invention would be useful for patients undergoing repeated treatments to the same location of the body, such as patients receiving radiation treatment for certain types of cancer and to the therapists providing such treatments. Other treatments or methodologies that require location and identification of a marked area on a human or animal and that would benefit from having a substantially invisible marking are also contemplated by the present invention. In addition, as shown in FIG. 4, the excitation source 42 may be integrated in or connected to the medical equipment 44 such that a proper excitation source is always readily available when providing a corresponding medical treatment. Such a configuration may be referred to generally as having the excitation source integrated within the medical treatment equipment.

An exemplary methodology for preparation and application of a tissue marking solution in accordance with the principles of the present invention that includes multi-photon excitation materials or pre-energized materials (explained herein) is as follows. The exemplary embodiment described herein utilizes the multi-photon materials referenced above from Sunstone, Inc. The materials utilized, are termed nano-sunstones having a 50-200 nanometer diameter and doped Y₂0₃, with rare earth materials that provide special absorption and emission characteristics. Such materials may be ordered to be excited with 980 nm radiation, however, it would be understood that the excitation frequency would vary according to the frequency of the excitation device intended to be utilized.

The multi-photon excitation materials are mixed with a carrier substance, or vehicle, such as Witch Hazel which can be purchased at a local pharmacy. A mixture is created of 1 part sunstones product with 2 parts Witch Hazel forming a paste (e.g., 30 grams of sunstones with 60 ml of Witch Hazel). The area to which the marking is to be applied should be cleaned with a topical antiseptic (isopropyl alcohol for example). Conventional medical marking equipment similar to that of tattoo equipment may be utilized for application purposes, such as for example, a seven needle configuration in a quill-pen type manor and used to apply the mark to the skin. Application of the marking solution will thus embed the multi-photon materials into the skin. As would be understood, post-treatment of the area may take place with a vitamin E cream and application of appropriate bandaging.

The marked location will generally be visible for a period of time, for example a number of days or weeks depending on the manner in which the patient heals. Then the marker will blend with the skin, being either invisible or substantially less visible than a pigmented marker. The marker will be substantially permanent and will produce visible light when irradiated with a specified IR wavelength (e.g., a standardized 980 nm source) dependant upon the specifications of the excitation materials that were utilized.

Other mixtures and or solutions may also be utilized in order to embed the excitation materials into a skin surface. These can include, for example, alcohol-based mixtures that include glycerin and or propylene additives.

As described earlier, the term “invisible” as used herein means not visible to the naked eye or substantially less visible than a pigmented tattoo under typical room lights. Depending upon the color of the skin, the marker or the associated tissue response may be detectable to the naked eye but less visible than a pigmented tattoo. It would be understood that in some cases that application of a medical marker in accordance with the present invention may cause hypo or hyper-pigmentation, tiny scarring, keyloiding or other reasons for the area being detectable to the naked eye. These conditions are understood to be contemplated within the meaning of an invisible medical marker in accordance with the present invention. Additionally, the term “permanent” means lasting long enough for a patient to proceed through at least one set of radiation treatments or for a for a number of years thereafter, for example.

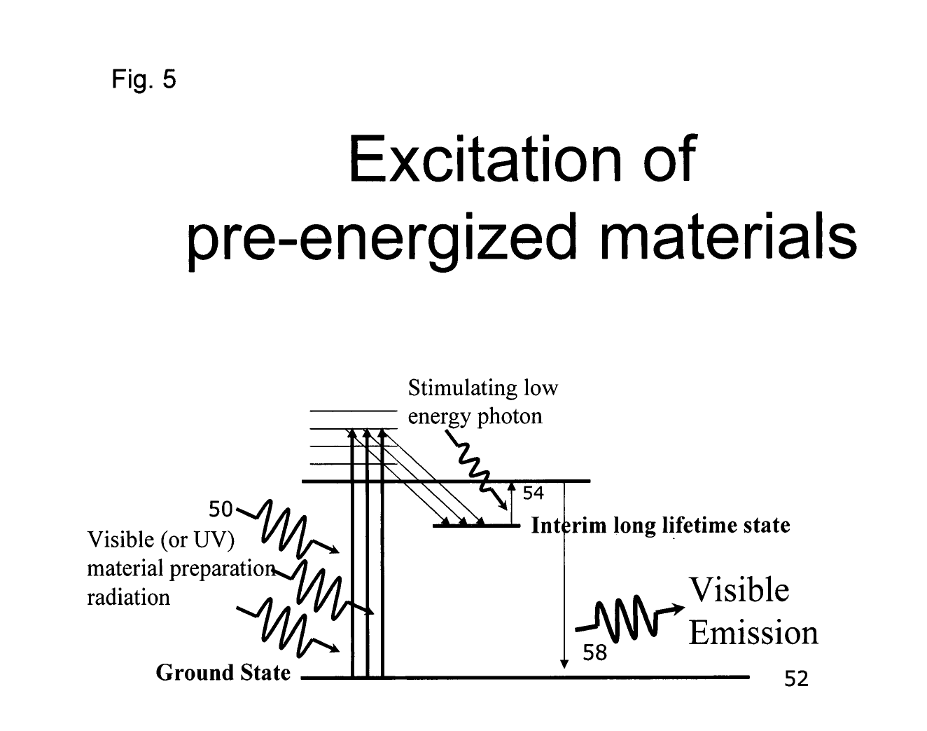

Other materials capable of producing a visible emission upon receiving energy stimulation are pre-energized materials such as storage-type phosphor materials available, for example, from maxmax.com of Carlstadt, N.J. Referring to FIG. 5, such materials become initially energized upon receiving material preparation radiation 50 from an energy source such as ultraviolet radiation, which elevates the state of the material from the ground state 52. Once energized, an interim long lifetime state 54 is available for a period of time upon which stimulating energy such as infrared radiation causes a visible emission 48 from the material. The present invention additionally contemplates use of those marking solutions that are activatable by means of either fluorescence or phosphorescence. Here, the active agent in the marking solution is able to absorb a higher energy visible or UV photon in order to produce a visible emission. As would be understood, fluorescence and phosphorescence are examples of visible emission being produced with a frequency down-conversion methodology, wherein absorption of a higher energy visible or UV photon produces the visible emission. A drawback for utilization of such methodologies, however, is that activation may require exposure to UV light sources, where prolonged exposure over time is known to have possible harmful effects. Further, there may be unintended UV activation sources such as black lights in public places, etc., that are able to cause such markings to become visible at unwanted times. Such methodologies may also be considered to be advantageous in some circumstances, since once exposed to ultraviolet light, the fluorescence and phosphorescence based activations will continue visible emission for a period of time, which can be controlled by varying the make-up of the activatable agent.

As would be understood, the ink or marking solution utilized to apply the invisible medical marking of the invention may utilize a number of different formulations. One exemplary embodiment may include ZnS:Cu phosphor, for example, which is used in glow-in-the-dark cosmetic creams frequently used for Halloween make-ups. U.S. Pat. No. 6,470,891 to Carroll, the teachings of which are incorporated herein by reference, describes a cosmetic tattoo that is invisible to the eye until it is exposed to ultraviolet (UV) radiation. The Carroll patent does not, however, teach or suggest that such a tattoo may have application for medical tissue markings, where a medical treatment is applied to the medical marking area after it becomes visible. Further, the Carroll patent does not contemplate irradiating the area for the purpose of identifying the area for a medical application. Thus the present invention is distinguishable in that it provides for the making of a medical marking, the later irradiating of the medical marking, and thereafter performing a medical procedure based on the located marking.

The marking solution utilized for the application of the invisible medical tissue markers may also include photochromic compounds normally invisible to the naked eye, but which become visible upon exposure to sunlight or other sources of ultraviolet radiation (UV). This is similar to the fluorescence methodology described above. The photochromic compounds are mixed in a non-pigmented or clear carrier that is substituted for the normal pigmented ink or dye that is used to apply the medical marker. The marking thus remains invisible to the naked eye until it is exposed to UV.

Examples of suitable photochromic compounds are described in U.S. Pat. Nos. 5,581,090 and 5,730,961, the disclosures of which are incorporated in full herein by reference. As noted in the '090 and '961 patents, photochromic substances are known in the art for their utilization in optics as storage media or as a means to detect UV, such as, e.g., the photochromic ultraviolet detector disclosed in the '090 patent. Among the large family of photochromic compounds are the spiropyrans and spiroxazines groups of molecules. These molecules are known for their property of changing from clear to a variety of colors and shades. Normally appearing as colorless, these spiro-compounds undergo a photochemical transformation to intensely colored form when exposed to UV. The '961 patent, in particular, discloses a material which remains clear until exposed to UV and which then exhibits color and thus becomes visible. The active chemical disclosed in the '961 patent is identified as a photochromic substance such as spiropyrans or spiroxazines molecules.

A carrier for the photochromic compounds could comprise an invisible skin-marking ink such as that made by Sirchie Finger Print Laboratories of Youngsville, N.C., under the name “Invisible Skin Marking Ink #743”, and described more fully in U.S. Pat. No. 5,878,155. However, other suitable commercially available, transparent, non-toxic carriers could be used to embed the photochromic compounds in the skin.

The medical markers of the invention may be applied in a similar fashion to those permanent visible markers found in the prior art. As an example, the medical markers may be applied using commercial tattooing equipment, such as rotary and wrench tattooing equipment.

The foregoing description merely illustrates the principles of the invention. It will thus be appreciated that those skilled in the art will be able to devise various arrangements, which, although not explicitly described or shown herein, embody the principles of the invention, and are included within its spirit and scope. For example, the markings as taught by the present invention may also be used for identification purposes, wherein an individual, class of individuals or even animal is marked with an identifier such as a name, number or other marking. The classes of people may include military persons, children and criminals, such as sex offenders, etc. as examples, to name a few although other classes are contemplated within the spirit of the invention. Although the application of the medical markings are described herein in connection with surface markings similar to that of tattoos, other application methodologies for introducing or embedding the active compounds into the body or skin surface are also contemplated, such as surgical applications. Furthermore, all examples and conditional language recited are principally intended expressly to be only for instructive purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure. Many other modifications and applications of the principles of the invention will be apparent to those skilled in the art and are contemplated by the teachings herein. Accordingly, the scope of the invention is limited only by the claims.

What is claimed is:

1. A method of marking an exterior skin surface for marking purposes comprising the steps of:
locating an area on said skin surface to be marked; and
marking said area with a substantially colorless marking solution suitable for embedding in said skin surface, said marking solution including one or more active compounds that appear substantially colorless and are substantially invisible to the naked eye until exposed to an excitation source, said active compounds causing a frequency change to light emitted from said excitation source and directed thereto, thereby further causing visible light to be emitted from said active compounds while said excitation source is directed at said marked area.

2. The method of claim 1, wherein said marking is a medical marking.

3. A method as claimed in claim 1, wherein the active compounds are frequency upconverting materials.

4. A method as claimed in claim 3, wherein the active compounds are rare-earth based frequency upconverting materials.

5. The method of claim 1, wherein the active compounds are pre-energized materials.

6. The method of claim 1, further including the step of applying said excitation source to said skin surface in order to make said marking visible.

7. The method as claimed in claim 6, further including the step of performing a medical procedure utilizing said marked area having been identified by application of said energy source.

8. The method of claim 6, further including the step of temporarily marking said area of said visible marking resulting from exposure to said excitation source.

9. The method of claim 1, wherein the active compounds are pre-energized materials.

10. The method of claim 1, wherein the excitation source is selected from the group consisting of: a source of ultraviolet radiation, a source of infrared radiation, a lamp and an LED.

11. The method of claim 1, wherein the excitation source is a laser.

12. The method of claim 1, wherein the marking is for identification purposes.

13. The method of claim 1, wherein the marking is applied to a class of people.

14. A system for marking an exterior skin surface comprising:
a substantially colorless marking solution suitable for embedding in said skin surface;
said marking solution including one or more active compounds that appear substantially colorless and are substantially invisible to the naked eye until exposed to an excitation source, said active compounds causing a frequency change to light emitted from said excitation source and directed thereto, thereby further causing visible light to be emitted from said active compounds while said excitation source is directed at said marked area.

15. The system of claim 14, wherein the active compounds are frequency upconverting materials.

16. The system of claim 15, wherein the active compounds are rare-earth based frequency upconverting materials.

17. The system of claim 14, wherein the active compounds are pre-energized materials.

18. The system of claim 12, wherein said marking solution is operable for use as a medical marker.

19. The system of claim 18, wherein said medical marker is activated for use in a medical procedure.

20. The system of claim 14, further including said excitation source for directing at said skin surface in order to make said marking visible.

21. The system as claimed in claim 20, wherein the excitation source is selected from the group consisting of: a source of ultraviolet radiation, a source of infrared radiation, lamp and an LED.

22. The system of claim 20, wherein the excitation source is a laser.

23. The system of claim 20, wherein the excitation source is integrated with a medical treatment device.

24. The method of claim 14, wherein the marking is for identification purposes.

25. A method of utilizing a medical marking comprising the steps of:
generally locating a marked area, said area marked with a marking solution embedded in tissue, said solution including one or more active compounds that appear substantially colorless and are substantially invisible to the naked eye until exposed to an excitation source, said active compounds causing a frequency change to light emitted from said excitation source thereby altering the frequency of the light by frequency up conversion or frequency down conversion and emitting visible radiation;
exciting said active compounds to cause visible radiation to emit from said marked area, thereby allowing exposure of said marked area for purposes of medical treatment.

24. The method of claim 25 further including performing a medical procedure utilizing said marked area.

26. A method of claim 25, wherein: the active compounds are rare-earth based frequency upconverting materials.

27. The method of claim 25, wherein the active compounds are pre-energized materials.

28. A method of claim 25, wherein the active compounds are selected from the group consisting of fluorescent and phosphorescent materials.

29. The method of claim 28, wherein the excitation source is selected from the group consisting of: a source of ultraviolet radiation, a source of infrared radiation, lamp and an LED.

30. The method of claim 28, wherein the marking is for identification purposes.

31. A method of marking comprising:
locating a position on an exterior skin surface to be marked; and
marking the exterior skin surface with a nearly invisible marking, said marking being for the purposes of detecting said position subsequent to exposure to illumination of a specific frequency range.

32. The method of claim 31 wherein said marking is embedded into said skin surface.

33. A method of claim 31, wherein said marking includes an active compound selected from the group consisting of frequency upconverting materials and pre-energized materials.

34. A method of claim 31, wherein the active compounds are selected from the group consisting of fluorescent and phosphorescent materials.

35. The method of claim 31, wherein the illumination is selected from the group consisting of: a source of ultraviolet radiation, a source of infrared radiation and an LED.

36. The method of claim 31, wherein said marking is a medical marking.

37. A method of utilizing a substantially invisible skin marking, comprising:
generally locating a position on an exterior skin surface marked with a nearly invisible marking, said marking being for the purposes of detecting said position subsequent to exposure to illumination of a specific frequency range; and
illuminating said exterior skin surface to allow exposure of said marking for purposes related to medical treatment.

38. A method of claim 37, wherein said marking includes an active compound selected from the group consisting of frequency upconverting materials and pre-energized materials.

39. A method of claim 37, wherein the active compounds are selected from the group consisting of fluorescent and phosphorescent materials.
